(19) United States
(12) Patent Application Publication
(10) Pub. No.: US 2021/0322200 A1
(43) Pub. Date: Oct. 21, 2021

(54) SYSTEM AND METHOD FOR PREPARING HOLLOW CORE CRANIAL REMODELING ORTHOSES

(71) Applicant: HEADSTART MEDICAL Ltd., Bumaby (CA)

(72) Inventor: Jason Shane GOODNOUGH, Port Moody (CA)

(21) Appl. No.: 16/632,516

(22) PCT Filed: Mar. 20, 2019
(86) PCT No.: PCT/CA2019/050338

§ 371 Date: Sep. 24, 2020

Related U.S. Application Data
(60) Provisional application No. 62/678,221, filed on May 30, 2018.
(60) Provisional application No. 62/645,704, filed on Mar. 20, 2018.

(51) Int. Cl. A61F 5/058; B29C 64/393; B33Y 80/00; B33Y 30/00; B33Y 50/02; G16H 50/50; G16H 70/20; G16H 30/20; G16H 10/60; G05B 19/4099

(52) U.S. Cl. CPC A61F 5/05891; B29C 64/393; B33Y 80/00; B33Y 30/00; B33Y 50/02; B29C 64/153; G16H 70/20; G16H 30/20; G16H 10/60; G05B 19/4099; G16H 50/50

(57) ABSTRACT

A system for creating cranial remolding orthoses comprises a controller in data communication with an additive fabricator and a scanner. The controller has access to a database of mappings of nonstandard cranial shapes to desired cranial shapes, to cranial scan data for a patient with an existing cranium shape, and to patient-specific information about the patient. The controller has design software for determining from the scan data a development path from the existing cranium shape to a desired cranium shape, the development path comprising a plurality of development path stages. Fabrication software in the controller allows it to instruct the fabricator for fabricating a cranial remodeling orthoses corresponding to the development path without requiring a physical model. The orthoses comprise a monolithic hollow core shell of thickness varying according to the desired remodeling of the cranium, an inner soft liner; and a fastener disposed for mounting the orthoses on the cranium of the patient.

### BACKGROUND OF THE INVENTION

### Field of the Invention

This present invention relates to the medical field as exemplified by IPC class A61, in particular class A61F 5/00 directed to orthopedic methods or devices for non-surgical treatment of bones or joints. More particularly, the invention is directed to IPC class A61F 5/058 (Splints) in addressing an apparatus and method for creating hollow core cranial remodeling orthoses for correcting deformations of the infantile human cranium.

### Description of the Related Art

The head of an infant may often be misshapen as a result of the head being molded during the birth process. Generally, the head gradually assumes a normal shape within the first six weeks of life. However, infants exhibit an abnormal head shape due to persistent external forces and that does not return to entirely normal shape without intervention. One example of such a condition is the so-called flat head syndrome, known in the medical field as Deformational Brachycephaly (DB). Deformational Brachycephaly is a specific condition in which the skull is flattened at the back, thereby rendering the skull shorter than normal and vaulted excessively high in the back region. Deformational Plagiocephaly (DP) is the condition in which the skull is distorted into asymmetric right versus left sided proportions. Deformational Scaphocephaly (DS) is the condition in which the skull is excessively long and narrow. These various conditions may manifest in many different and complex deformations of the cranium. They may be the result of premature birth, cervical anomalies, birth trauma, a restrictive intrauterine environment, sleeping position, or a combination of these or other factors.

Surgery is often the selected treatment for non-deformational cranial deformities called craniosynostoses, but surgery does not lend itself to treating deformational cranial asymmetries. These difficulties have spurred the use of external orthotic devices to provide non-invasive, non-surgical correction of cranial abnormalities and for post-surgical cranial remodeling and protection in select cases. Such orthoses have been developed to correct these cranial abnormalities and employ the advantages offered by the soft, growing skull of an infant. The orthosis device may be employed to restrain growth in some protruding areas of the skull and allows for growth where needed elsewhere on the skull.

Standardized orthoses have been used extensively in treating the above conditions. However, given the unique condition of each individual infant, this is a field better served by custom orthoses created to correct the specific condition of the child. Non-invasive custom orthosis devices have been described in the prior art. They are typically of plastic construction comprised of a semi-rigid outer shell bonded to medium hardness polyethylene foam inner lining. The devices are typically fabricated by taking a Plaster of Paris impression of the patient's head, from which is made a mold or cast that is a replica of the patient's head. A second or corrected head mold is then fashioned by filling in areas on the first mold to produce a mold which is of the desired head shape. The orthosis device is then created over the corrected mold. The correction included in forming the corrected mold is based on clinical experience and anthropomorphic measurements. The modifications are based in large part upon specialist experience and skill.

Depending on the age of the patient at the start of treatment, the degree of severity of cranial abnormality, and the rate of cranial growth, the prior art treatment may require more than one orthosis device to be created during the course of treatment. It may also be necessary to change the initial orthosis design during the course of treatment.

More recently, systems have been presented in which a three-dimensional optical scanner is employed to generate input patient cranial data. This data is then provided to a suitable computer having access to a database containing anthropomorphic data such as age, sex, race, height, weight and similar information which is of significance in determining an appropriate desired head shape. The computer is also provided with pertinent patient-specific information. The computer further includes software to utilize the anthropomorphic data in conjunction with the patient specific data and the scan data to design an appropriate orthosis device. The computer commands a milling machine to produce an exact positive mold of the patient's preferred head shape. The clinician may then utilize the positive mold to manually produce a cranial remodeling orthosis. Alternatively, the orthosis data may be directly provided to a molding machine which directly produces the orthosis. Despite the improvements over earlier methods, the method still results in heavy and expensive orthoses and repeated consults with associated costs. The weight and thickness of the orthosis is a significant problem in that the muscular and skeletal development of infants may be such as to render the weight and bulk of the orthosis a hindrance to the developing patient.

One object of the present invention is to use the efficiencies of novel technology to improve patient access to cranial remodeling. A further object of the present invention is to drastically reduce the weight, thickness, and heat retention of cranial orthoses in order to improve safety and infant comfort. Yet a further object of the present invention is to provide a system and method of fabricating orthoses less reliant on the skills of individual orthotists or clinicians.

### SUMMARY OF THE INVENTION

The invention involves designing a cranial remolding orthoses based on patient development to guide the early growth of an infant's skull. The skull configuration of the patient is determined by examination, with the prothesis designed to inhibit growth in abnormally large areas, and allow expansion into a desired growth pattern. Further, the mass and encapsulation of the orthoses minimizes the negative effects of the orthoses on the patient's development. Areas desired to be inhibited receive pressure, while areas desired to be grown are provided free space for expansion. The growth area includes ventilation holes, and the entire orthoses is manufactured by additive methods that are strong and flexible, allowing for the application of appropriate pressure while minimizing the weight of the orthoses to minimize impact on the infant. Lower weight and ventilation mitigate against negative events for the patient and also make the orthoses more comfortable so that it is more likely to be worn for a sufficient treatment period.

In a first aspect, a system is provided for creating cranial remolding orthoses for remodeling a cranium, the system comprising: an additive fabricator; a database comprising for different patient-specific information a collection of mappings of desired cranial shapes to existing cranial shapes; prior patient cranial scan data for a patient cranium having and existing patient cranium shape; a controller in communication with the fabricator and with access to the prior cranial scan data and the database and, the controller having design software which when executed determines from the prior patient cranial scan data a development path from the existing patient cranium shape to a desired patient cranium shape; and fabrication software which when executed communicates instructions to the fabricator for fabricating a remodeling orthoses for the patient cranium.

The system may further comprise a scanner for obtaining the prior patient cranial scan data from a scan of the patient. The scanner may be a three-dimensional optical scanner. The additive fabricator may be arranged to fuse a thermoplastic polymer material into a shaped object under digital control. The additive fabricator may in particular be a fused filament fabricator, a stereolithography system, a continuous liquid interface production system, a selective laser sintering system, or selective heat sintering system.

In a further aspect, a method is provided for creating a cranial orthosis, the method comprising: Providing a controller having: a database a collection of mappings of desired cranial shapes to existing cranial shapes; design software which when executed determines from patient cranial scan data a development path from an existing patient cranium shape to a desired patient cranium shape; and fabrication software which when executed communicates instructions to a fabricator to fabricate an object; providing an additive fabricator capable of fabricating a three-dimensional object based on instructions from the controller; providing to the controller patient-specific information about a patient; providing to the controller patient cranial scan data from a scan of a cranium of the patient having an existing cranial shape; executing the design software to automatically determine a desired patient cranium shape from the cranial scan data based on the patient-specific information and the collection of mappings; determining corresponding to the desired patient cranium shape a cranial remodeling orthosis for achieving the desired patient cranium shape; and executing the fabrication software to send instructions to the additive fabricator to fabricate a cranial orthoses for the patient.

In a further aspect, a method is provided for creating a cranial remodeling orthoses corresponding to a cranium of a patient having an existing cranial shape, the method comprising: providing a controller having: a database comprising a collection of mappings of desired cranial shapes to existing cranial shapes; design software which when executed determines from cranial scan data of a patient a development path from an existing cranium shape to a desired cranium shape; and fabrication software which when executed communicates instructions to a fabricator to fabricate an object; providing an additive fabricator in communication with the controller, the fabricator capable of fabricating a three-dimensional object based on instructions from the controller; providing to the controller patient-specific information about the patient; providing to the controller patient cranial scan data describing the existing cranial shape; executing the design software to determine from the cranial scan data based on the patient-specific information and the collection of mappings a cranial orthoses designed to facilitate cranial development from the existing cranium shape to a desired cranium shape; dividing the cranial development path into a corresponding shape of a cranial remodeling orthosis for achieving the corresponding desired shape; and fabricating the cranial orthoses of the patient by executing the fabrication software to provide fabrication instructions to the additive fabricator.

Providing to the controller patient cranial scan data may comprise performing a scan of the existing cranium. Fabricating the cranial orthoses may comprise forming the orthoses from a thermoplastic material. Fabricating the cranial orthoses may comprise forming the orthoses from thermoplastic filament by fused filament fabrication. Fabricating the cranial orthoses may comprise forming the orthoses from thermoplastic powder by one of selective laser sintering and selective heat sintering. Fabricating the cranial orthoses may comprise forming the orthoses from a photopolymer. Forming the orthoses may comprise forming the orthoses from a photopolymer by one of stereolithography and continuous liquid interface production.

In a further aspect, a cranial remodeling orthosis is provided for a cranium of a patient, the orthosis comprising a hollow core outer shell of a thickness that spatially varies according to scan data of the cranium. The orthosis may further comprise: an inner soft liner disposed to provide contact between the shell and the cranium; and a fastener disposed for mounting the orthosis on the cranium. The shell may be composed of one of a solidified photopolymer and a thermoplastic material. The thermoplastic material may be one of polycarbonate, polyvinyl alcohol, polyactic acid, and acrylonitile butadine styrene. The photopolymer may be one of an acrylate and an epoxy resin.

The outer shell may be a single monolithic unit. The single monolithic unit may comprise at least one ventilation portion. The outer shell may comprise a single monolithic unit comprising at least one opening shaped to receive a ventilation unit; and at least one corresponding ventilation unit shaped to fit into the at least one opening.

### BRIEF DESCRIPTION OF THE DRAWINGS

The above mentioned and other features and objects of this invention, and the manner of attaining them, will become more apparent and the invention itself will be better understood by reference to the following description of an embodiment of the invention taken in conjunction with the accompanying drawings, wherein:

FIG. 1 is a schematic drawing of a system for creating by additive fabrication a plurality of cranial remolding orthoses for remodeling a cranium in a corresponding plurality of stages.

FIG. 2 is a flow chart for a method of creating by additive fabrication a cranial remolding orthosis for remodeling a cranium.

FIG. 3 is a flow chart for a method of creating by additive fabrication a plurality of cranial remolding orthoses for remodeling a cranium in a corresponding plurality of stages.

FIG. 4 is a drawing of a cranial remodeling orthosis device made by the system and method of the present invention.

FIGS. 5-7 are graph drawings of cranial measurements providing insight on the type of orthosis for a particular patient.

FIGS. 8-11 are photographic representations of an embodiment of orthosis for one type of patient characteristics.

Corresponding reference characters indicate corresponding parts throughout the several views. Although the drawings represent embodiments of the present invention, the drawings are not necessarily to scale and certain features may be exaggerated in order to better illustrate and explain the present invention. The flow charts and screen shots are also representative in nature, and actual embodiments of the invention may include further features or steps not shown in the drawings. The exemplification set out herein illustrates an embodiment of the invention, in one form, and such exemplifications are not to be construed as limiting the scope of the invention in any manner.

### DESCRIPTION OF EMBODIMENTS OF THE PRESENT INVENTION

The embodiments disclosed below are not intended to be exhaustive or limit the invention to the precise form disclosed in the following detailed description. Rather, the embodiments are chosen and described so that others skilled in the art may utilize their teachings.

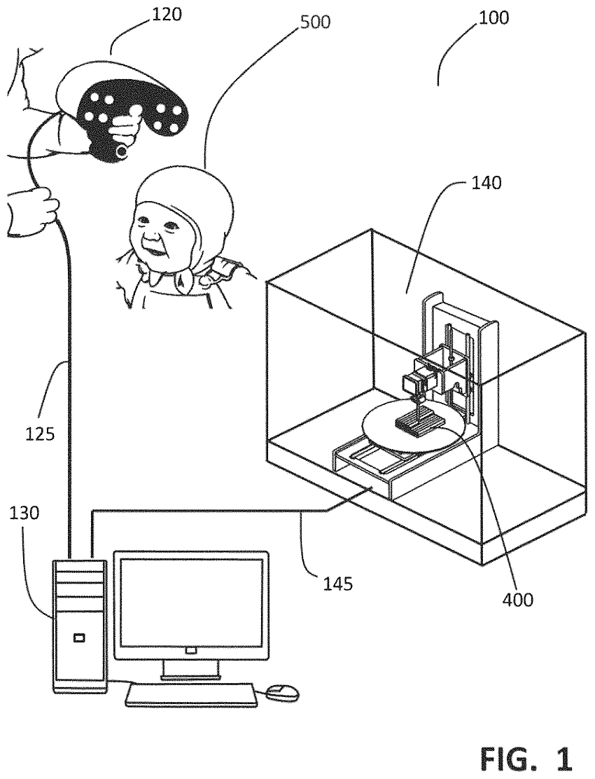

In the schematic drawing of FIG. 1, a system 100 for creating cranial remolding orthoses for remodeling an infant cranium comprises an additive fabricator 140. Additive fabricator 140 may be a so-called “three-dimensional printing” system employing suitable polymers as forming material. Suitable additive fabricators include, for example without limitation, laser-based stereolithography (SLA) systems employing photopolymers, continuous liquid interface production (CLIP) systems also using photopolymers, fused filament fabrication (FFF) systems employing continuous fiber thermoplastic materials, selective laser sintering (SLS) systems employing powdered thermoplastic materials, and selective heat sintering (SHS) of powdered thermoplastic materials.

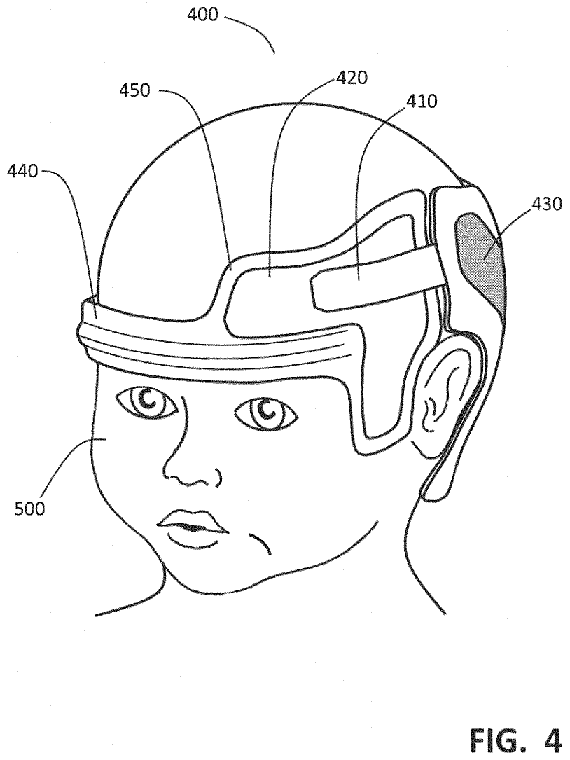

Fused filament fabrication, also referred to as Fused deposition Modeling (FDM), has become a very popular and cost-effective “3D printing” technology. In this technology, a continuous filament of a thermoplastic material is fed from a coil of the material through a moving, heated printer extruder head. Molten material is forced out of a nozzle of the print head and is deposited on the growing workpiece. The head is moved, under computer control, to define the product shape. Other additive additive manufacturing technologies may be used to create the customized cranial orthoses, such as deposition printing and the like, and in fact conventional injection molding could be used to manufacture the orthoses although with current technologies would be expensive. In FIG. 1, additive fabricator 140 is schematically shown fabricating an additive cranial remolding orthosis 400, which will be discussed in more detail at the hand of FIG. 4. In general, fabricators suitable for the present purpose are arranged to fuse a thermoplastic polymer material into a shaped object under digital control.

Additive fabricator 140 is controlled by controller 130 via control link 145. Control link 145 may be any suitable link capable of transmitting control data from controller 130 and may be wired or wireless. In some embodiments controller 130 may generate a suitable instruction data file and the file may then be transferred to additive fabricator 140 by means of suitable portable data storage media, including without limitation magnetic disc media and digital data storage media such as, for example, Universal Serial Bus (USB) “flash/jump drives”, portable external hard drives, portable external solid state memory drives, memory cards, and the like.

Controller 130 may be, for example without limitation, a computer or digital workstation with a suitable processor, operating random access memory, data storage means including for example hard disk drives and/or solid state drives, digital communication ports, data input devices including for example a keyboard and a digital mouse, portable data storage media, and a display monitor. Controller 130 may also be capable of and configured for accessing data on portable data storage media, including without limitation magnetic disc media and digital data storage media such as, for example, Universal Serial Bus (USB) “flash/jump drives”, portable external hard drives, portable external solid state memory drives, memory cards, and the like.

Controller 130 has access to a database comprising a collection of mappings of desired cranial shapes to existing cranial shapes. There may be more than one potential desired cranial shape for every existing cranial shape and in general the final desired cranial shape may depend on age, sex, race, height, weight and similar information.

Controller 130 may be separately provided with patient-specific information and the patient specific-information may be employed to determine a particular desired cranium shape. Beyond identity information about the patient, the patient-specific information may comprise anthropomorphic information specific to the patient. This may include the patient's sex, race, age, weight, height, and other features characterizing the patient, such as neck strength, skull age, and preferred sleeping positions, and may include health information. In some embodiments, the controller 130 may be in communication with a database in a public health system in order to obtain the patient specific information.

Generally, a patient would be identified as a candidate for cranial molding around three months after birth, and the physician or other medical professional would refer the patient to a clinic for an orthoses. During an initial patient visit, the clinic would measure the cranial development by either direct measurement or cranial scan, e.g., a photo or x-ray. For example, a patient may be so scanned with a small thin cap over the cranium, the cap having a visual and/or radiographic presence for detection of the other periphery of the patient's current cranial development. The determination of the desired cranial shape from a given existing cranial shape may be done automatically by cranium orthosis design software stored on the data storage means of the controller 130, loaded into its memory, and executed by its processor. In this process the determination may be automatically guided by the patient-specific information. Alternatively, a clinician may visually match orthoses shapes with measured cranial development. When employing the database of mappings, the cranium orthosis design software may treat the patient-specific information as parameters controlling the mappings. In other embodiments, the determination may be based at least in part on human decision-making with reference to the patient-specific information.

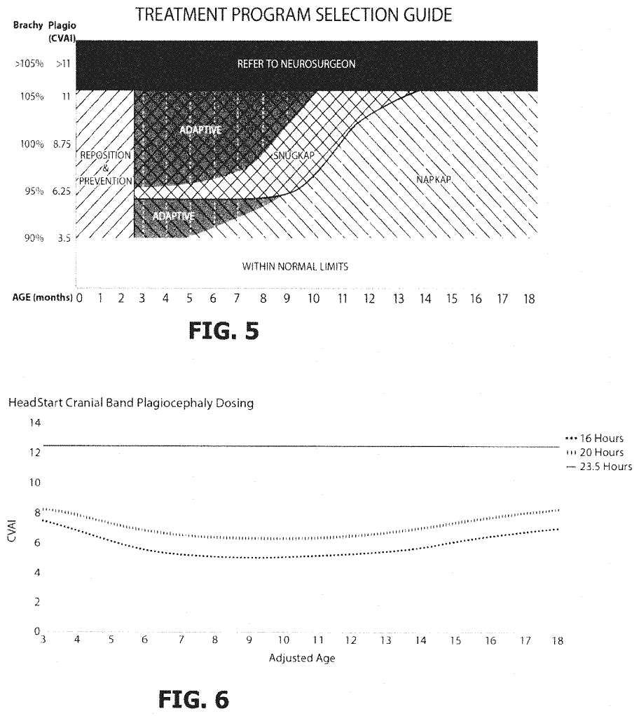

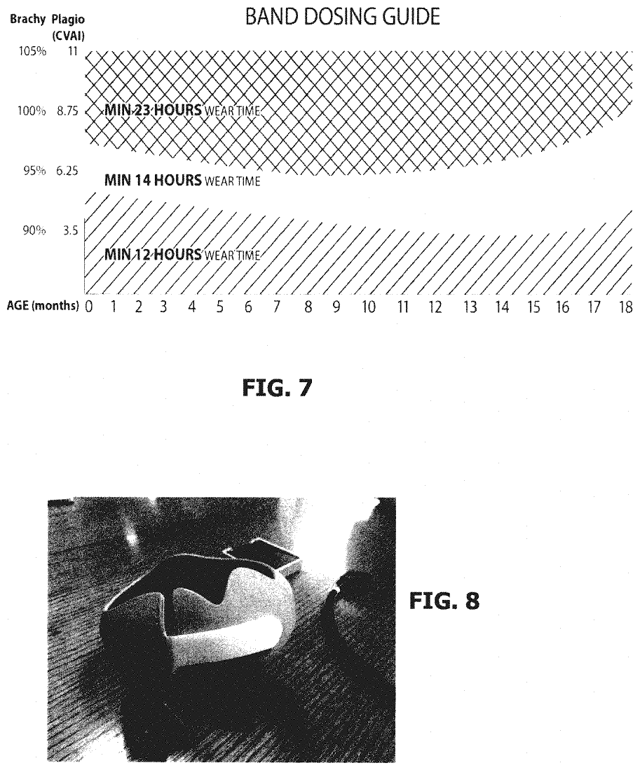

FIG. 5 shows graphically how an individual patient after 3 months may be classified as either within normal limits (no orthoses needed), having a cranial shape that may be positively affected by a cranial orthoses. The amount of time per day that a patient should have the orthoses in place would be determined by the severity of the cranial development and the age of the patient. FIGS. 6 and 7 show graphs mapping the amount of time each day that the patient should wear the orthoses. Such a patient would have a second check-up, typically six or eight weeks after having a first orthoses was provided and after wearing for the prescribed amounts of time, and the cranial development again checked. The second visit would involve another measurement and/or scan of the cranial development, and a second orthoses would then be created and provided to the patient. Ideally, a patient would have a single cranial orthoses and by a second check-up return to cranial development within normal limits, but often the cranial repositioning takes several months and possibly several different cranial orthoses.

The cranium orthosis design software is further capable of designing a cranial development path from an existing cranial shape to a desired cranial shape, based on scan data of the existing cranial shape and on the patient-specific information. Such a path of three-dimensional development of the cranium may have along its passage of progress several waypoints of which at least one may be selected, either by human intervention or automatically by the cranium orthosis design software. The at least one waypoint divides the cranial development path into a plurality of cranial development path stages each having a corresponding end cranial shape. The cranium orthosis design software is further capable of designing cranial orthoses based on the existing cranial shape, the end cranial shapes, and the cranial development path stages. The term “scan data” is employed in the present specification to describe data obtained from a scan of a relevant portion of an anatomy, being in the present case the cranium of the patient, in a format that may be interpreted by the cranium orthosis design software employed by controller 130 as described below. The term “scan” is employed in the present specification to describe any form of scan capable of reducing to suitable scan data the shape in three dimensions of the relevant portion of the anatomy, such as visual indications like photographs, as well as x-rays, magnetic resonance imaging, ultrasonic imaging, and similar scanning technologies.

Controller 130 may be provided with fabrication software for converting the cranial orthosis designs into file formats and instructions that can be interpreted by the fabricator 140 and for controlling fabricator 140 via the instructions. In particular, controller 130 is capable of providing to the fabricator 140 instructions to fabricate all of the plurality of cranial orthoses for the entire cranial development path.

FIG. 1 further schematically shows a patient 500 in a scanner 120, which in this case is a three-dimensional optical scanner. In general the scanner 120 may be any scanner capable of providing suitable scan data describing an existing cranium shape of the patient 500 in three dimensions in a format that may be interpreted by the cranium orthosis design software employed by controller 130. In FIG. 1, scanner 120 is shown linked to controller 130 by scan data link 125, which may be wired or wireless. However, in other embodiments scan data may be manually transferred from scanner 120 to controller 130 by means of a suitable portable data storage media, including without limitation magnetic disc media and digital data storage media such as, for example, Universal Serial Bus (USB) “flash/jump drives”, portable external hard drives, portable external solid state memory drives, memory cards, and the like.

Operationally, the controller 130 may be a controller that is supplied with the additive fabricator 140. Since the scanner equipment 120 is expensive and has to serve many different medical care end users not necessarily focusing on orthoses, the controller 130 and additive fabricator 140 may usefully be housed together, though there may be implementations in which the additive fabricator 140 also has to multi-task and may need to be housed distant from the controller 130. As a consequence, in the general case the system may comprise the controller 130, the scanner 120 and the additive fabricator 140 and they may be independently housed remote from one another and arranged to communicate with one another via wired or wireless links, or via portable data storage media.

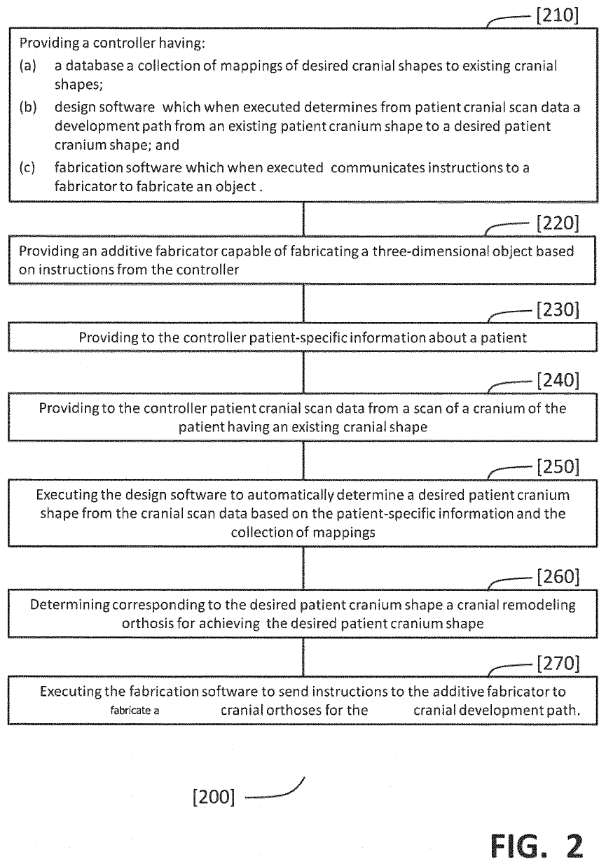

In another aspect, described at the hand of FIG. 2, a method [200] is provided for making a cranial orthosis, the method comprising providing [210] a controller 130 having: a database a collection of mappings of desired cranial shapes to existing cranial shapes; design software which when executed determines from patient cranial scan data a development path from an existing patient cranium shape to a desired patient cranium shape; and fabrication software which when executed communicates instructions to a fabricator to fabricate an object; providing [220] an additive fabricator 140 capable of fabricating a three-dimensional object based on instructions from the controller 130; providing [230] to the controller patient-specific information about a patient 500; providing [240] to the controller patient cranial scan data from a scan of a cranium of the patient 500 having an existing cranial shape; executing [250] the design software to automatically determine a desired patient cranium shape from the cranial scan data based on the patient-specific information and the collection of mappings; determining [260] corresponding to the desired patient cranium shape a cranial remodeling orthosis for achieving the desired patient cranium shape; and executing [270] the fabrication software to send instructions to the additive fabricator 140 to fabricate acranial orthoses for the desired cranial development. The phrase “automatically determine” is used here to describe a situation in which no physical models or casts of the existing cranium are made in order to manually derive from those models or casts a desired cranium shape. In order to reduce the involvement of human experts, the human expertise is accommodated into the design software.

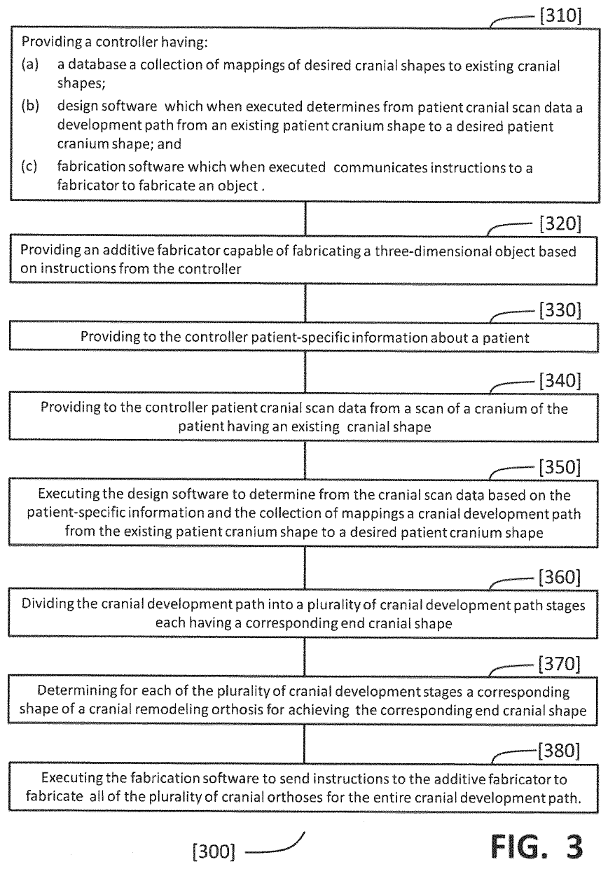

In another aspect, a method [300] described at the hand of the flow chart of FIG. 3 is provided for creating a plurality of cranial remodeling orthoses corresponding to a plurality of development path stages of the cranium of a single patient 500, the method comprising: providing [310] a controller 130 having: a database comprising a collection of mappings of desired cranial shapes to existing cranial shapes; design software which when executed determines from a patient cranial scan data a development path from an existing patient cranium shape to a desired patient cranium shape; and fabrication software which when executed communicates instructions to a fabricator 140 to fabricate an object; providing [320] an additive fabricator 140 in communication with the controller 130 and capable of fabricating a three-dimensional object based on instructions from the controller 130; providing [330] to the controller 130 patient-specific information about the single patient 400; providing [340] to the controller 130 patient cranial scan data from a scan of the cranium of the single patient 500 having an existing cranial shape; executing [350] the design software to determine from the cranial scan data based on the patient-specific information and the collection of mappings a cranial development path from the existing patient cranium shape to a desired patient cranium shape; dividing [360] the cranial development path into a plurality of cranial development path stages each having a corresponding end cranial shape; determining [370] for each of the plurality of cranial development stages a corresponding shape of a cranial remodeling orthosis for achieving the corresponding end cranial shape; and executing [380] the fabrication software to send instructions to the additive fabricator to fabricate all of the plurality of cranial orthoses 400 for the entire cranial development path of the single patient 500. The determining [370] corresponding shapes of cranial remodeling orthoses may comprise automatically determining the corresponding shapes. As already explained, no physical models are involved in the process.

In a further aspect an additive cranial remodeling orthosis 400 is provided as shown in FIG. 4. The cranial remodeling orthosis 400 comprises: a hollow core orthotic outer shell 450 of thickness varying according to a desired remodeling of the cranium of the patient 500, an inner soft liner (obscured in FIG. 4) for contact with the cranium; and a fastener 410 disposed for mounting the orthosis 400 on the cranium of the patient 500. The hollow core orthotic outer shell 450 may be fabricated by additive fabricator 140 of FIG. 1 under control of controller 130 for remodeling the cranium of patient 500 through one cranial development stage to an end cranial shape associated with the particular cranial development stage as determined in steps [260], [270] and [280] of the method of FIG. 3. In one embodiment, the fastener may be held in place by hook and loop fasteners, such as a Velcro® fastener (VELCRO is a registered trademark of Velcro Industries B.V. LIMITED LIABILITY COMPANY NETHERLANDS Castorweg 22-24 Curacao NETHERLANDS). In further embodiments, other means may be employed to hold the fastener 410 in place.

Given that additive cranial remodeling orthosis 400 has to extend around the cranium in order to be usefully worn, band 440 may extend around the cranium of the patient 500 in order to ensure adequate compressive force in the compressive portions 420 when the orthosis is applied to the cranium of the 500. Unlike prior art non-additive orthoses that are made as a singular attempt to correct a cranium, additive cranial remodeling orthosis 400 does not need to be bulky and may be of low weight, because by the method described at the hand of FIG. 2 no removal of material of the orthosis 400 is required along the development path of the cranium.

Orthotic outer shell 450 may be composed of a single additively depositable thermoplastic material. The single additively depositable thermoplastic material may be any one of, or a mix of a polycarbonate, polyactic acid (PLA), polyvinyl alcohol (PVA), Copolyester (CPE), an aliphatic polyamide including Nylon, thermoplastic polyurethane (TPU), high impact polystyrene (HIP), softer and less brittle forms of polyetheylene terephthalate (PET) and acrylonitile butadine styrene (ABS). Polycarbonate and polyactic acid (PLA) are favored materials. In other embodiments, corresponding to the use of stereolithography (SLA) or continuous liquid interface production (CLIP), the orthotic outer shell 450 may be composed of a single solidified photopolymer. The single solidified photopolymer may be one of an acrylate and an epoxy resin.

Orthotic outer shell 450 may comprise compressive portions 420 and ventilation portions 430. The compressive portions 420 provide the basic orthotic function while the ventilation portions 430 improve the comfort of the patient via ventilation. The presence of the ventilation portions 430 also prevents pressure on the cranium from comparatively uncompressible surfaces in the environment during sleep. The ventilation portions 430 may comprise structures that provide rigidity and dimensional stability to orthosis 400 whilst using very little material. Due to the low material density in the ventilation portions 430 they substantially reduce the overall weight of the orthosis 400. In practice the inventors have found that the hollow core construction of orthosis 400 leads to a reduction of the orthosis weight down to as little as 30% of the weight of comparable non-hollow core orthoses. This is of profound benefit to the infant user during general motion when the momentum of the head is of great concern as regards safety and enabling unimpeded gross motor development in the treated children.

In some embodiments, the ventilation portions 430 may be fabricated monolithically with the rest of the orthosis 400. In other embodiments, the ventilation portions 430 may be fabricated separately from the rest of the orthosis 400. In yet further embodiments, the ventilation portions 430 may be fabricated separately from the rest of the orthosis 400 as standard components of fixed shape and size. The rest of the orthosis 400, including specifically the compressive portions 420, may be fabricated on a custom basis to provide standardized openings in non-compressive areas of orthosis 400 into which the ventilation portions 430 may be fitted. This latter approach reduces the unit cost of the ventilation portions and the amount of custom fabrication that has to be done.

The hollow core of orthosis 400 may have any internal structure that provides suitable integrity of the structure and dimensional stability of orthosis 400. It increases the resistance of orthosis 400 to torsion and other deformation while also reducing its weight. A variety of such low density and high rigidity structures, for example without limitation geometrical, honeycomb, tubular, and otherwise, are well known in many parts of Industry, including Packaging, Construction, Furniture and other, and will not be dwelt on further here.

Three-dimensional inkjet machines and thermal extrusion deposition devices using a variety of fiber materials have become more available and their cost has dropped drastically. Along with this, the optimization of use of materials that these machines provide has made it possible to fabricate the orthosis using a minimum of materials so that the orthosis will have minimal weight. The orthotic outer shell 450 may further vary by design in its matrix structure, for example in the compressive portions 420 as compared with non-compressive portions 430 so as to optimize cranial corrective function, materials use, and weight. The general thickness of orthosis 400 is considerably reduced from that of prior art orthoses.

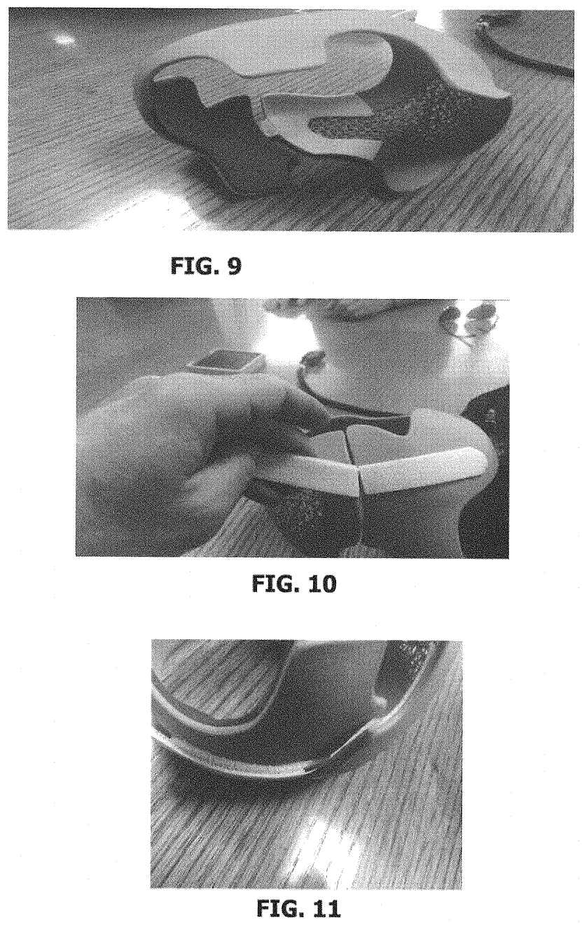

In prior art orthoses, the outer shell is usually of substantially constant thickness, and is then filled on the inside with a cranium correction material that is fashioned to provide the cranial corrective function. As the cranium proceeds along its growth path, cranium correction material is then removed to accommodate the cranium. In the present additive cranial remodeling orthosis 400 there is no removal of material involved and it is the additively fabricated outer shell that performs the cranial correction function. The liner on the inside of orthosis 400 may serve merely as a softer pad against the cranium and has no requirement to vary in thickness for any functional reason. Various photos of one embodiment of orthoses are shown in FIGS. 8-11, showing many of the particular features discussed above.

Returning now to the methods of FIG. 2 and FIG. 3 minimization of materials use and minimization of weight may be factors included in the design software of the controller 130 and may be employed in determining practical cranial development path waypoints and stages, the associated end cranial shapes, and thereby the required shape of the associated cranial remodeling orthoses 400.

The principle at work in this approach is that of dividing up the cranial development path into stages and designing a plurality of orthoses 400, one for each stage, each comprising a hollow core orthotic shell 450 of thickness varying according to a desired remodeling of the cranium of the patient 500, an inner soft liner for contact with the cranium; and a fastener 410 disposed for mounting the orthosis 400 on the cranium of the patient 400. None of these additive cranial remodeling stage orthoses 400 are required to be as bulky or heavy as prior art orthoses, because no material is removed from them during the growth of the cranium and material is only used where needed. Also, only one initial consult between patient (or guardians of patients) is required for the required plurality of custom orthoses to be fabricated. Although in the typical case the patient will visit the clinic for second and subsequent check-ups for the stages of cranial repositioning, in some embodiments the patient may transmit scans of the current stage of cranial development to a suitably configured computing system and have software for creating a manufacturing program for a suitable additive fabricator so that the patient would not need to be physically moved to receive further customized orthoses.

While this invention has been described as having an exemplary design, the present invention may be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

1. A system for creating cranial remolding orthoses for remodeling a cranium, the system comprising:
an additive fabricator;
a database comprising a collection of mappings of desired cranial shapes to existing cranial shapes;
prior patient cranial scan data for a patient cranium having an existing patient cranium shape;
a controller in communication with the fabricator and with access to the prior cranial scan data and the database and, the controller having
design software which when executed determines from the prior patient cranial scan data a development path from the existing patient cranium shape to a desired patient cranium shape, the development path comprising a plurality of development path stages; and
fabrication software which when executed communicates instructions to the fabricator for fabricating a plurality of cranial remodeling orthoses corresponding to the plurality of development path stages.

2. The system of claim 1, further comprising a scanner for obtaining the prior patient cranial scan data from a scan of the patient, optionally a three-dimensional optical scanner.

3. The system of claim 1, wherein the additive fabricator is arranged to fuse a thermoplastic polymer material into a shaped object as specified by the fabrication software.

4. The system of claim 1, wherein the additive fabricator is one of a fused filament fabricator, a stereolithography system, a continuous liquid interface production system, a selective laser sintering system and a selective heat sintering system.

5. The system of claim 1, wherein the the additive fabricator is a fused filament fabricator.

6-8. (canceled)

9. A method for creating a cranial remodeling orthosis, the method comprising:
providing a controller having:
a database comprising a collection of mappings of nonstandard cranial shapes to desired cranial shapes;
design software which when executed determines from patient cranial scan data a development path from an existing patient cranium shape to a desired patient cranium shape; and
fabrication software which when executed communicates instructions to a fabricator to fabricate an object;
providing an additive fabricator capable of fabricating a three-dimensional object based on instructions from the controller;
providing to the controller patient-specific information about a patient;
providing to the controller patient cranial scan data from a scan of a cranium of the patient having an existing cranial shape;
executing the design software to automatically determine a desired patient cranium shape from the cranial scan data based on the patient-specific information and the collection of mappings;
determining corresponding to the desired patient cranium shape a cranial remodeling orthosis for achieving the desired patient cranium shape; and
executing the fabrication software to send instructions to the additive fabricator to fabricate a cranial orthosis for the patient.

10. The method of claim 9 wherein providing to the controller patient cranial scan data comprises performing a scan of the existing cranium.

11. The method of claim 9 wherein fabricating the cranial orthosis comprises one of forming the orthosis from a thermoplastic material, forming the orthosis from thermoplastic filament by fused filament fabrication, forming the orthosis from thermoplastic powder by one of selective laser sintering and selective heat sintering, forming the orthosis from a photopolymer, and forming the orthosis s from a photopolymer by one of stereolithography and continuous liquid interface production.

12-15. (canceled)

16. A cranial remodeling orthosis for a cranium of a patient, the orthosis comprising a hollow core outer shell of a thickness that spatially varies according to scan data of the cranium of the patient.

17. The cranial remodeling orthosis of claim 16, further comprising an inner soft liner disposed to provide contact between the shell and the cranium; and a fastener disposed for mounting the orthosis on the cranium.

18. The orthosis of claim 16, wherein the shell is composed of a thermoplastic material, and optionally the thermoplastic material is one of polycarbonate, polyvinyl alcohol, polyactic acid, and acrylonitile butadine styrene.

19. The orthosis of claim 16, wherein the shell is composed of a photopolymer, and optionally the photopolymer is one of acrylate and an epoxy resin.

20. The orthosis of claim 16, wherein the outer shell is a single monolithic unit.

21. The orthosis of claim 20, wherein the single monolithic unit comprises at least one ventilation portion.

22. The orthosis of claim 21, wherein the ventilation portion comprises a structure allowing the passage of air through the ventilation portion.

23. The orthosis of claim 16, wherein the outer shell comprises:
a single monolithic unit comprising at least one opening shaped to receive a ventilation unit; and
at least one corresponding ventilation unit shaped to fit into the at least one opening.

24. The orthosis of claim 16, wherein the core of the shell comprises a high rigidity and low density structure.

25. The orthosis of claim 24, wherein the high rigidity and low density structure is one of a honeycomb structure and a tubular structure.

26. The orthosis of claim 16, wherein the thickness of the shell further spatially varies according to patient specific information.

27. The cranial remodeling orthosis of claim 16, wherein the hollow core is structured to provide a weight of the orthosis that is less than the weight of the orthosis when the core is solid and made from the same material, optionally as low as 30% of the weight of the orthosis when the core is solid and made from the same material.

28. A method for creating a plurality of cranial remodeling orthoses corresponding to a plurality of development path stages toward a desired cranium shape of the cranium of a patient having an existing cranial shape, the method comprising:
automatically determining for each of the cranial development path stages a corresponding shape of a cranial remodeling orthosis for achieving a corresponding end cranial shape; and
fabricating the cranial orthoses for the entire cranial development path of the single patient by executing fabrication software to provide fabrication instructions to an additive fabricator.

29. The method of claim 28, further comprising:
providing a controller having:
a database comprising a collection of mappings of nonstandard cranial shapes to desired cranial shapes;
design software which when executed determines from cranial scan data of a patient the development path from the existing cranium shape to the desired cranium shape; and
fabrication software which when executed communicates instructions to a fabricator to fabricate an object;
providing the additive fabricator in communication with the controller, the fabricator capable of fabricating a three-dimensional object based on instructions from the controller;
providing to the controller patient-specific information about the single patient;
providing to the controller patient cranial scan data describing the existing cranial shape;
executing the design software to determine from the cranial scan data based on the patient-specific information and the collection of mappings a cranial development path from the existing cranium shape to the desired cranium shape, the cranial development path comprising:
a sequential plurality of cranial development path stages; and
corresponding to each of the cranial development path stages an end cranial shape.

30. The method of claim 29, wherein providing to the controller patient cranial scan data comprises performing a scan of the existing cranium.

31. The method of claim 29, wherein fabricating the cranial orthoses comprises one of forming the orthoses from a thermoplastic material, forming the orthoses from thermoplastic filament by fused filament fabrication, forming the orthoses from thermoplastic powder by one of selective laser sintering and selective heat sintering, and forming the orthoses from a photopolymer by one of stereolithography and continuous liquid interface production.

32. The method of claim 29, wherein fabricating the cranial orthoses comprises forming the orthoses to each have a weight substantially less than the weight of a cranial orthosis fabricated based on a single development path stage between the existing cranium shape and the desired cranium shape.

33. A cranial remodeling kit for adjusting an existing cranial shape along a cranial development path to a desired cranial shape, the kit comprising a plurality of additively fabricated cranial remolding orthoses corresponding to a sequential plurality of cranial development path stages along the cranial development path.

34. The cranial remodeling kit of claim 33, wherein each of the plurality of orthoses has a weight substantially less than the weight of a single orthosis, each of the plurality of orthoses being additively fabricated based on a single development path stage between the existing cranium shape and the desired cranium shape.

35. The cranial remodeling kit of claim 34, wherein each orthosis has an outer shell comprising a structured hollow core.

36. The cranial remodeling kit of claim 34, wherein each orthosis has a core comprising one of honeycomb structure and a tubular structure.

37. The cranial remodeling kit of claim 34, wherein the shell of each orthosis is composed of a thermoplastic material, and optionally the thermoplastic material is one of polycarbonate, polyvinyl alcohol, polyactic acid, and acrylonitile butadine styrene.

38. The cranial remodeling kit of claim 34, wherein the outer shell of each orthosis is composed of a photopolymer, and optionally the photopolymer is one of acrylate and an epoxy resin.

39. The cranial remodeling kit of claim 34, wherein the outer shell of each orthosis is a single monolithic unit.

40. The cranial remodeling kit of claim 39, wherein the single monolithic unit comprises at least one ventilation portion.

41. The cranial remodeling kit of claim 40, wherein the ventilation portion comprises a structure allowing the passage of air through the ventilation portion.

42. The cranial remodeling kit of claim 34, wherein each orthosis has an outer shell comprising:
a single monolithic unit comprising at least one opening shaped to receive a ventilation unit; and
at least one corresponding ventilation unit shaped to fit into the at least one opening.

43. The cranial remodeling kit of claim 34, wherein each orthosis has a shell with a core, and the core of the shell comprises a high rigidity and low density structure.

44. The cranial remodeling kit of claim 43, wherein the high rigidity and low density structure is one of a honeycomb structure and a tubular structure.

45. The cranial remodeling kit of claim 34, wherein each orthoses has an outer shell with a thickness that spatially varies according to patient specific information.
